(19) United States
(12) Patent Application Publication
(10) Pub. No.: US 2010/0098640 A1
(43) Pub. Date: Apr. 22, 2010

(54) Multidentate Pyrone-Derived Chelators for Medicinal Imaging and Chelation

(76) Inventors: Seth M. Cohen, San Marcos, CA (US); David T. Puerta, Rockland, MA (US)

Correspondence Address: JONES DAY, 222 EAST 41ST ST, NEW YORK, NY, 10017, US

(21) Appl. No.: 11/922,454

(22) PCT Filed: Jun. 20, 2006
(86) PCT No.: PCT/US2006/024010

§ 371 Date: Dec. 29, 2009

Related U.S. Application Data
(60) Provisional application No. 60/692,431, filed on Jun. 20, 2005.

(51) Int. Cl. A61K 49/06; C07D 315/00; A61K 31/35; C07F 15/00; A61P 43/00

(52) U.S. Cl. 424/9.361; 549/417; 514/460; 549/210

(57) ABSTRACT

Provided herein are chelating agents and metal chelates that are useful in diagnostic and therapeutic applications. The uses of metal chelates provided herein include their use as contrast agents in medical imaging modalities, such as magnetic resonance imaging (MRI).

### RELATED APPLICATION DATA

This application claims priority to U.S. provisional application Ser. No. 60/692,431, filed Jun. 20, 2005, entitled “MULTIDENTATE PYRONE-DERIVED CHELATORS FOR MEDICINAL IMAGING AND CHELATION” Cohen et al. The disclosure of the above referenced application is incorporated by reference herein.

### FIELD

Provided herein are chelating agents and metal chelates. The metal chelates are useful in diagnostic and therapeutic applications. In certain embodiments, the metal chelates are useful as contrast agents in diagnostic imaging, such as magnetic resonance imaging (MRI), X-ray, nuclear radiopharmaceutical imaging, ultraviolet/visible/infrared light, and ultrasound.

### BACKGROUND

The technique of superimposing linear field gradients to the static magnetic field of a nuclear magnetic resonance (NMR) experiment to obtain three-dimensional images of an object (Lauterbur, P. C., Nature 1973, 190) is known as magnetic resonance imaging (MRI). Whereas conventional X-rays show skeletal structure, MRI enables the acquisition of high resolution, three-dimensional images of the distribution of water in vivo. This powerful diagnostic tool is invaluable in the detection of a wide variety of physiological abnormalities including tumors; lesions, and thrombosis. Additionally, recent advances in dynamic MRI open up the exciting possibility of real-time imaging of biochemical activity (“A New Generation of In Vivo Diagnostics,” MetaProbe, 2000). MRI has many advantages over other imaging techniques, the most notable being that MRI does not require the use of ionizing radiation or radioactive isotopes and provides superb imaging quality, as well as “real time” imaging capabilities.

The medical utility of MRI is enhanced through the administration of contrast agents prior to the scan, which alters the relaxation times of protons in the vicinity of the agent, increasing the degree of contrast between healthy and diseased tissue. The use of contrast agents is increasingly popular in medical protocols, with some 30-35% of MRI scans now acquired with the aid of a contrast agent (Caravan, P. E. et al., Chem. Rev. 1999, 99, 2293; Aime, S. B. et al., E. Acc. Chem. Res. 1999, 32, 941). A number of paramagnetic metal ions (Mn²⁺, Fe³⁺, Gd³⁺) and superparamagnetic metal clusters (various ferric oxide particles) have been studied for use as contrast agents.

Several new contrast agents are currently under development, which are designed to be more site-specific, facilitating, for example, detailed images of cardiovascular features (Lauffer, R. B., Magn. Reson. Med. 1991, 22, 339). Additionally, recent reports have demonstrated that contrast agents can detect the presence of enzymes and metal cations (Moats, R. A. F. et al., Angew Chem., Int. Ed. Engl 1997, 36, 726; Li, W. F. et al., J. Am. Chem. Soc. 1999, 121, 1413).

Certain of the clinically accepted contrast agents are based upon a gadolinium complex of a poly(aminocarboxylate) ligand, e.g., the gadolinium chelates of DTPA, DOTA, DO₃A and DTPA-BMA. These agents are extracellular agents that distribute non-specifically throughout the plasma and interstitial space of the body. A typical use of such agents is in the detection of tumors in the brain.

The image enhancing capability of available agents is far lower than the optimal values predicted by theory (Aime, S. B. et al., Coord. Chem. Rev., 321: 185-6 (1999)). The relatively low image enhancing properties of current contrast agents requires injection of gram quantities in order to obtain satisfactory contrast in the resulting image. There continues to be a need for contrast agents of increased image enhancement capacity and corresponding enhanced water proton relaxivity.

### SUMMARY

Provided herein are chelating ligands and metal chelates. The metal chelates are useful in diagnostic and therapeutic applications. In certain embodiments, the metal chelates are useful as contrast agents in diagnostic imaging, such as magnetic resonance imaging (MRI), x-ray, nuclear radiopharmaceutical imaging, ultraviolet/visible/infrared light, and ultrasound imaging.

The metal chelates provided herein are water-soluble paramagnetic metal chelates containing a metal ion complexed with one or more ligands. In one embodiment, the metal chelates are kinetically and thermodynamically stable to minimize any toxicity associated with the free metal ion or the chelating ligand. In certain embodiments, the metal chelates are neutral in order to reduce osmotic shock during intravenous administration. In certain embodiments, the metal chelates have more than one inner sphere water molecule coordinated to the metal center (q>1) in order to increase relaxivity. The paramagnetic chelates provided herein have high water exchange rates, and correspondingly high proton relaxation rates such that they are effective MRI contrast agents.

In certain embodiments, the metal chelates provided herein are thermodynamically stable metal complexes of pyrone-based ligands. The complexes contain a polypodal framework that creates a binding cavity for the metal ion. The chelating structure of the metal chelates is stabilized by strong hydrogen bonds during metal complexation and the ligands use hard oxygen donors that have a high affinity for strong Lewis acidic metals. In certain embodiments, the metals used in the compounds provided herein include, but are not limited to ions of lanthanides and actinides. In one embodiment, the metal is Ga, Dy, Fe, Mn, Pu or U. In certain embodiment, in the compounds provided herein, a metal ion is coordinated by the oxygen donor atoms of the chelating agents.

In certain embodiments, the ligands for use in the metal chelates provided herein contain one or more chelating units tethered together to a polypodal scaffold or a backbone. In certain embodiments, the chelating units in the ligands provided herein have formula I:

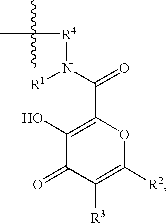

wherein R¹ is hydrogen, alkyl, alkenyl, alkynyl, aryl, heteroaryl, cycloalkyl, heterocyclyl or C(A)R⁵;

R² and R³ are each independently selected from hydrogen, alkyl, alkenyl, alkynyl, aryl, heteroaryl, cycloalkyl, heterocyclyl, C(A)R⁵, OR⁶ and NR⁷R⁸;

R⁴ is alkylene, alkenylene, alkynylene, cycloalkylene, arylene, heteroarylene or heterocyclylene group, where R⁴ is connected to a scaffold or a backbone that tethers together two or more chelating units to form the ligands provided herein;

A is O, S or NR⁷;

R⁵ is hydrogen, alkyl, alkenyl, alkynyl, aryl, heteroaryl, heteroarylium, cycloalkyl, heterocyclyl, halo, pseudohalo, OR⁶ or NR⁷R⁸;

R⁶ is hydrogen, alkyl, alkenyl, alkynyl, aryl, heteroaryl, heteroarylium, cycloalkyl, heterocyclyl;

R⁷ and R⁸ are each independently selected from hydrogen, alkyl, alkenyl, alkynyl, aryl, heteroaryl, cycloalkyl, heterocyclyl or R⁷ and R⁸ together with the nitrogen atom on which they are substituted form a heterocyclic or heteroaryl ring.

In certain embodiments, the metal chelates provided herein have formula II or IIA:

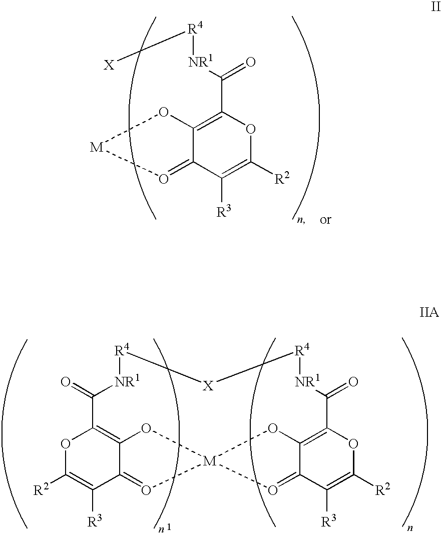

wherein X is a scaffold, M is a metal, n and n¹ are each independently 1-6 and other variables are as described elsewhere herein. The metal ions for use herein, include, but not limited to ions of Gd, Ga, Dy, Fe, Mn, Pu, and U.

Also provided herein are methods to prepare the metal chelates described herein. In the methods, the parameters that improve contrast ability of the compounds provided herein, including water residence lifetime and molecular weight, can be optimized to maximize relaxivity. The synthetic flexibility is important so that ligands containing tissue-specific, hydrophobic (to increase non-covalent protein binding and thereby τ_{R}), hydrophilic (to improve water solubility), or macromolecular components can be prepared. The synthetic pathways to the chelates herein provide for the facile incorporation of subunits that modify one or more properties of the chelates.

Also provided are pharmaceutically-acceptable derivatives, including salts, esters, enol ethers, enol esters, solvates, and hydrates of the compounds described herein. Further provided are pharmaceutical compositions containing the compounds provided herein and a pharmaceutically acceptable carrier. In one embodiment, the pharmaceutical compositions are formulated for single dosage administration.

Further provided is a method of performing contrast-enhanced magnetic resonance imaging on a patient. The method includes administering to the patient a compound provided herein in an amount sufficient to provide contrast enhancement, and acquiring a contrast enhanced MR image.

The chelating agents provided herein are also of use for binding radioisotopes utilized in nuclear medicine, gamma camera scintigraphy, and other medical applications.

Articles of manufacture are provided containing packaging material, a compound or composition provided herein which is useful as contrast agent, and a label that indicates that the compound or composition is useful as contrast agent.

### BRIEF DESCRIPTION OF FIGURES

FIG. 1 illustrates structure of [Fe(TREN-Me-MAM)] (left) and [Fe(TREN-Me-3,2-HOPO)] (right). Both ligands act as hexadentate chelators to the Fe3+ centers (orange spheres). Amide hydrogen atoms involved in stabilizing intramolecular hydrogen bonding are shown.

FIG. 2 shows electronic spectra of TRENMAM (solid line) and [Gd(TRENMAM)] (dotted line) recorded in water. The substantial changes in the λ max will be used to monitor the complexation reaction and thereby determine the thermodynamic stability of these complexes. T=25° C.

FIG. 3 is a structural diagram (50% probability ellipsoids) of [Fe(TREN-Me-MAM)] showing the anticipated ligand structure, metal coordination, and internal hydrogen bonding (bonds not explicitly shown) typical of these tripodal complexes. Hydrogen bonds exist between the amide nitrogen protons and deprotonated hydroxyl oxygen atoms (e.g. between N2 and O1). Hydrogen atoms have been omitted for clarity.

FIG. 4 provides comparison of the species distribution for (from top to bottom):

### TRENMAM, TREN-Me-MAM, [Gd(TRENMAM)], and Gd(TREN-Me-3,2-HOPO)].

FIG. 5 provides representative plot of competition titration data. The x-intercept indicates the difference in pGd between TRENMAM and DTPA.

[TRENMAM]=30.0 μM; [Gd]=30.0 μM; [DTPA]=3.00 μM-300 μM; pH=7.4; I=0.1 M KCl.

FIG. 6 provides 1/T₁ NMRD profiles of [Gd(TRENMAM)(H₂O)₂] and [Gd(TREN-Me-MAM)(H₂O)₂], at 310 K and pH 7.2.

FIG. 7 provides 1/Tᵢ NMRD profiles of [Gd(TRENMAM)(H₂O)₂] and [Gd(TREN-Me-MAM)(H₂O)₂], at 298 K and pH 7.2.

FIG. 8 demonstrates temperature dependence of the paramagnetic contribution to the water ¹⁷ONMR transverse relaxation rate (R₂ₚ) for [Gd(TRENMAM)(H₂O)₂], (represented by the solid circles in the figure, 0.019 M) and [Gd(TREN-Me-MAM)(H₂O)₂] (represented by the hollow circles in the figure, 0.013 M) at 2.12 T and pH 7.2.

### DETAILED DESCRIPTION

### A. Definitions

Unless defined otherwise, all technical and scientific terms used herein have the same meaning as is commonly understood by one of ordinary skill in the art to which the claimed subject matter belongs. All patents, applications, published applications and other publications are incorporated by reference in their entirety. In the event that there are a plurality of definitions for a term herein, those in this section prevail unless stated otherwise.

As used herein, MRI refers to magnetic resonance imaging, a procedure in which radio waves and a powerful magnet linked to a computer are used to create detailed pictures of areas inside the body. These pictures can show the difference between normal and diseased tissue. MRI makes better images of organs and soft tissue than other scanning techniques, such as CT or X-ray. MRI is especially useful for imaging the brain, spine, the soft tissue of joints, and the inside of bones.

As used herein, MRI contrast agent (MRI-CA) refers to compounds that are administered to a patient to enhance image quality obtained in MRI. The MRI-CA facilitate diagnosis by brightening the image in the immediate vicinity of the compound. The contrast agent localizes in a diseased tissue, such as cancerous, tissue and brightens the image and more clearly identifies the diseased, such as cancerous, tissues.

As used herein, scaffold refers to a backbone that tethers together two or more chelating units to form the ligands provided herein. Throughout the instant specification, the complexes provided herein are exemplified by embodiments in which one or more pyrone-based complexing group is attached to a linear, polyfunctional scaffold, forming a chelating agent with the correct geometry to complex a metal ion. The scaffolds for use in the complexes and chelating agents provided herein are exemplified by the use of TREN. The exemplary TREN scaffold is for clarity of illustration only and should not be interpreted as limiting the scope of the subject matter to a genus of chelating agents and complexes having a TREN backbone. Those of skill in the art will appreciate that a wide array of scaffold structures can be used as scaffold moieties in the compounds provided herein. For example, scaffolds of use herein can be linear, cyclic, saturated or unsaturated species. Some exemplary scaffold moieties are described elsewhere herein and in U.S. Pat. No. 6,846,915, which is incorporated by reference in its entirety.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, and cycloalkyl. Pharmaceutically acceptable enol esters include, but are not limited to, derivatives of formula C═C(OC(O)R) where R is hydrogen, alkyl, alkenyl, alkynyl, or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the claimed subject matter encompasses any racemic, optically active, polymorphic, or stereoisomeric form, or mixtures thereof, of a compound provided herein, which possesses the useful properties described herein, it being well known in the art how to prepare optically active forms and how to determine antiproliferative activity using the standard tests described herein, or using other similar tests which are will known in the art.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl and isohexyl. As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein, generally selected from Q1.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I. As used herein, pseudohalides or pseudohalo groups are groups that behave substantially similar to halides. Such compounds can be used in the same manner and treated in the same manner as halides. Pseudohalides include, but are not limited to, cyano, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1 chloro 2 fluoroethyl.

As used herein, “haloalkoxy” refers to RO in which R is a haloalkyl group.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or NYY′, where Y and Y′ are each independently hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C═C—, C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “substituted alkylene,” “substituted alkenylene,” “substituted alkynylene,” “substituted cycloalkylene,” “substituted cycloalkenylene,” and “substituted cycloalkynylene” refer to alkylene, alkenylene, alkynylene, cycloalkylene, cycloalkenylene and cycloalkynylene groups, respectively, that are substituted with one or more substituents, in certain embodiments one to three or four substituents, where the substituents are as defined herein.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 5 or 6 carbons.

As used herein, “heteroarylene” refers to a divalent monocyclic or multicyclic aromatic ring system, in one embodiment of about 5 to about 15 members where one or more, in certain embodiments 1 to 3, of the atoms in the ring system is a heteroatom, that is, an element other than carbon, including but not limited to, nitrogen, oxygen or sulfur.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted arylene,” “substituted heteroarylene” and “substituted heterocyclylene” refer to arylene, heteroarylene and heterocyclylene groups, respectively, that are substituted with one or more substituents, in certain embodiments one to three of four substituents, where the substituents are as defined herein.

Where the number of any given substituent is not specified (e.g., “haloalkyl”), there may be one or more substituents present. For example, “haloalkyl” may include one or more of the same or different halogens. As another example, “C₁₋₃alkoxyphenyl” may include one or more of the same or different alkoxy groups containing one, two or three carbons.

As used herein “subject” is an animal, such as a mammal, including human, such as a patient.

As used herein, the term “parenteral” includes subcutaneous, intravenous, intrathecal, intra-arterial, intramuscular or intravitreal injection, or infusion techniques.

The term “topically” encompasses administration rectally and by inhalation spray, as well as the more common routes of the skin and mucous membranes of the mouth and nose and in toothpaste.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944). Certain of the abbreviations used herein are as follow:

TREN=tris(aminoethyl)amine);

MAM=maltolamide

Me-MAM=methylmaltolamide

TRENMAM=tris(aminoethyl)amine)maltolamide;

TREN-Me-MAM=tris(aminoethyl)amine)methylmaltolamide

### B. Compounds

In certain embodiments, the chelating ligands provided herein have formula:

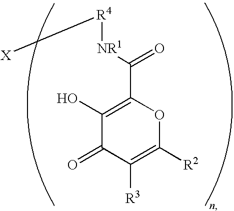

wherein X is a scaffold; n is 1-6;

R¹ is hydrogen, alkyl, alkenyl, alkynyl, aryl, heteroaryl, cycloalkyl, heterocyclyl or C(A)R⁵;

R² and R³ are each independently selected from hydrogen, alkyl, alkenyl, alkynyl, aryl, heteroaryl, cycloalkyl, heterocyclyl, C(A)R⁵, OR⁶ and NR⁷R⁸;

R⁴ is alkylene, alkenylene, alkynylene, cycloalkylene, arylene, heteroarylene or heterocyclylene group, where R⁴ is connected to a scaffold or a backbone that tethers together two or more chelating units to form the ligands provided herein; A is O, S or NR⁷;

R⁵ is hydrogen, alkyl, alkenyl, alkynyl, aryl, heteroaryl, heteroarylium, cycloalkyl, heterocyclyl, halo, pseudohalo, OR⁶ or NR⁷R⁸;

R⁶ is hydrogen, alkyl, alkenyl, alkynyl, aryl, heteroaryl, heteroarylium, cycloalkyl, heterocyclyl;

R⁷ and R⁸ are each independently selected from hydrogen, alkyl, alkenyl, alkynyl, aryl, heteroaryl, heteroarylium, cycloalkyl, heterocyclyl or R⁷ and R⁸ together with the nitrogen atom on which they are substituted form a heterocyclic or heteroaryl ring;

wherein R¹-R⁸ are each independently unsubstituted or substituted with one or more substituents, in one embodiment one to five substituents, in another embodiment one, two or three substituents, each independently selected from Q¹;

where Q¹ is hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano, isothiocyano, and each Q¹ is independently unsubstituted or substituted with one or more substituents, in one embodiment one, two or three substituents, each independently selected from Q²;

each Q² is independently hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano and isothiocyano.

In certain embodiments, R³ and R² are selected such that they enhance solubility of the metal chelate or can be further derivatized by methods known in the art to enhance solubility of the metal chelate or to append more complex functionalities. For example an alkylazido group for R² can be used for ‘click’ chemistry, a copper-mediated coupling with an acetylene group to generate a triazole ring in high yield and with a high tolerance for other functional groups. The alkylazido group can also be reduced to an amine group for potential functionalization via imine, amide, and alkylation reactions. A carboxyl group for R² can be selectively functionalized to couple groups via an amide or ester bond forming reaction. The R³ and R² groups are selected such that the resulting metal chelates have improved solubility, relaxivity, and targeting.

In certain embodiments, R³ is hydrogen or alkyl. In one embodiment, R³ is hydrogen. In certain embodiments, R² is hydrogen, optionally substituted alkyl or carboxy. In one embodiment, R² is hydrogen, hydroxyalkyl, azidoalkyl or carboxy. In other embodiment, R² is hydrogen, methyl, hydroxymethyl, azidomethyl or carboxy.

Exemplary Scaffolds

Any linear, polyfunctional scaffold, forming a chelating agent with the correct geometry to complex a metal ion can be used in the compounds provided herein. Those of skill in the art will appreciate that a wide array of scaffold structures can be used as scaffold moieties in the compounds herein. For example, scaffolds of use herein can be linear, cyclic, saturated or unsaturated species. Scaffolds for use in the metal chelates are known in the art, for example, see, U.S. Pat. No. 6,846,915. Some exemplary moieties are set forth below:

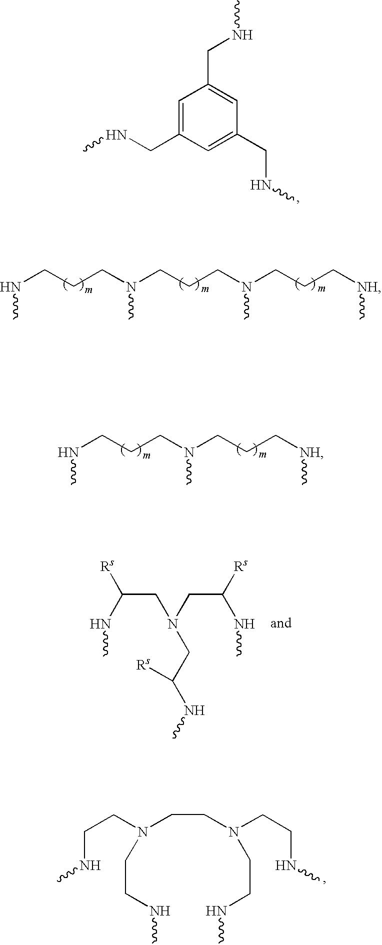

where m is 1 to 4 and R^{s} is hydrogen, alkyl or OR⁶. The scaffolds are linked to the chelating units of formula I.

In certain embodiments, provided herein are scaffolds that are functionalized with moieties that are available for interaction with a group on another molecule. Thus, the scaffolds can include reactive functional groups, in addition to those that are used to form the link between the scaffold and the chelating heterocyclic rings. The functional groups can be used to attach the ligand to another species, e.g., a targeting moiety, polymer, etc. In another exemplary embodiment, the water solubility of the complexes provided herein can be enhanced by the functionalization of the scaffold with an appropriate group (Hajela, et al., J. Am. Chem. Soc. 2000, 122, 11228).

Exemplary Ligands

In certain embodiments, the chelating ligands provided herein have formula III:

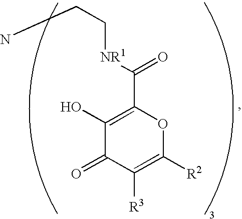

wherein the variables are as described elsewhere herein.

In certain embodiments, the chelating ligand provided herein is:

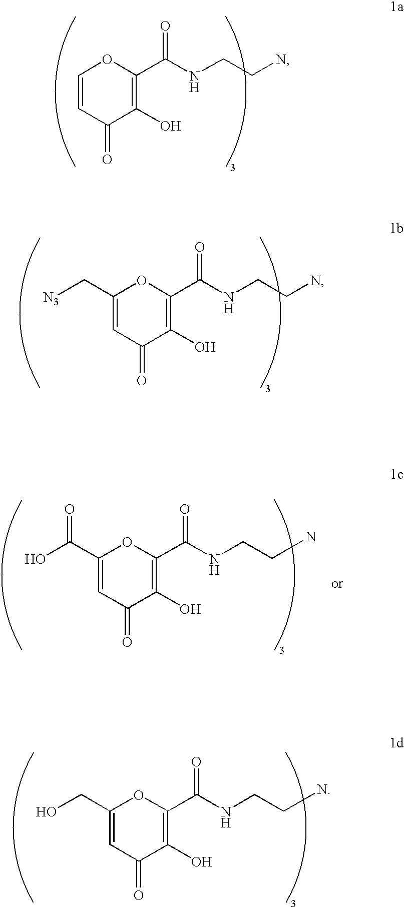

Exemplary Complexes

In certain embodiments, the metal chelates provided herein have formula II or IIA:

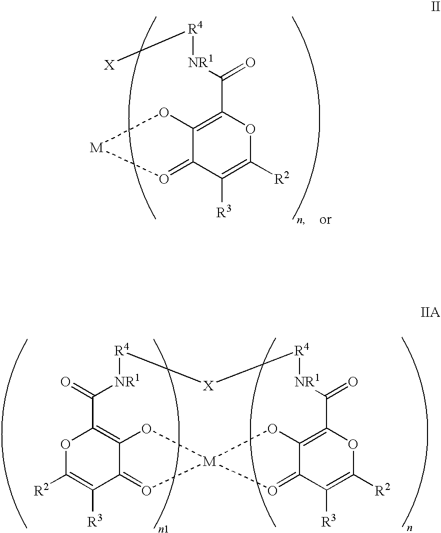

where M is selected from Gd, Ga, Dy, Fe, Mn, Pu, and U; n and n¹ are each independently 1 to 6 and the other variables are as described elsewhere herein.

In certain embodiments, the metal chelates provided herein have formula III:

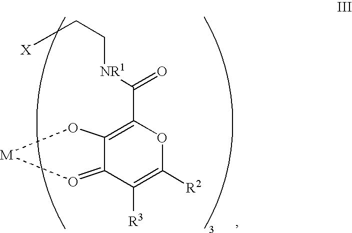

where the variables are as described elsewhere herein.

In certain embodiments, the metal chelates provided herein have formula VI:

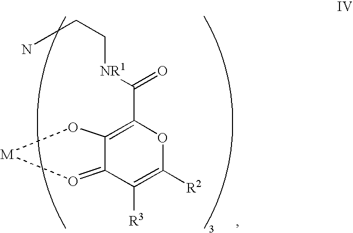

where M is selected from Gd, Ga, Dy, Fe, Mn, Pu, and U and the other variables are as described elsewhere herein. In certain embodiments, M is selected from Gd, Ga, and Fe.

In certain embodiments, the metal chelates provided herein have formula VII:

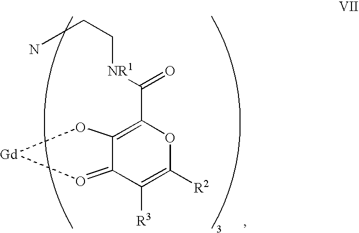

where the variables are as described elsewhere herein.

In certain embodiments, the metal chelates provided herein have formula VIII:

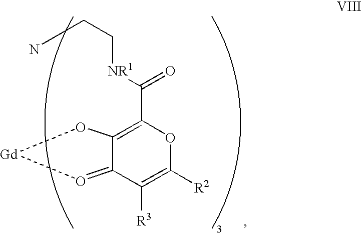

where the variables are as described elsewhere herein.

In certain embodiments, the metal chelate is:

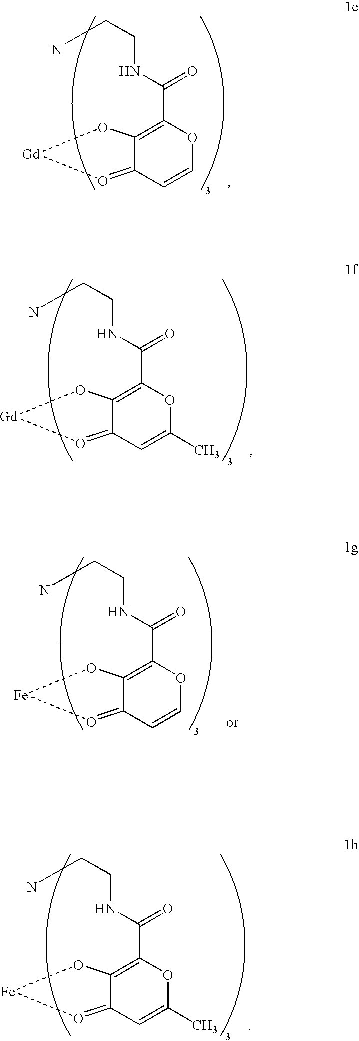

In certain embodiments, the metal chelate is:

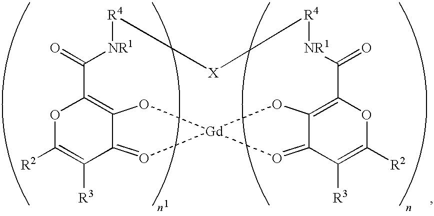

where the variables are as described elsewhere herein.

### C. Preparation of the Compounds

The following illustrations depict general preparations of compounds claimed herein and consist of reactions typically known to one skilled in the art of chemical synthesis. The substituents referred in the schemes are described elsewhere herein. Also it will be apparent to one skilled in the art that many of the products could exist as one or more isomers, that is E/Z isomers, enantiomers and/or diastereomers.

The precursors for each ligand for use herein are prepared by using the methods in the chemical literature. Exemplary synthetic routes for the precursor of certain of the ligands provided herein are described in the Examples. General methods for the preparation of TRENMAM and TREN-Me-MAM are illustrated in schemes 1 and 2.

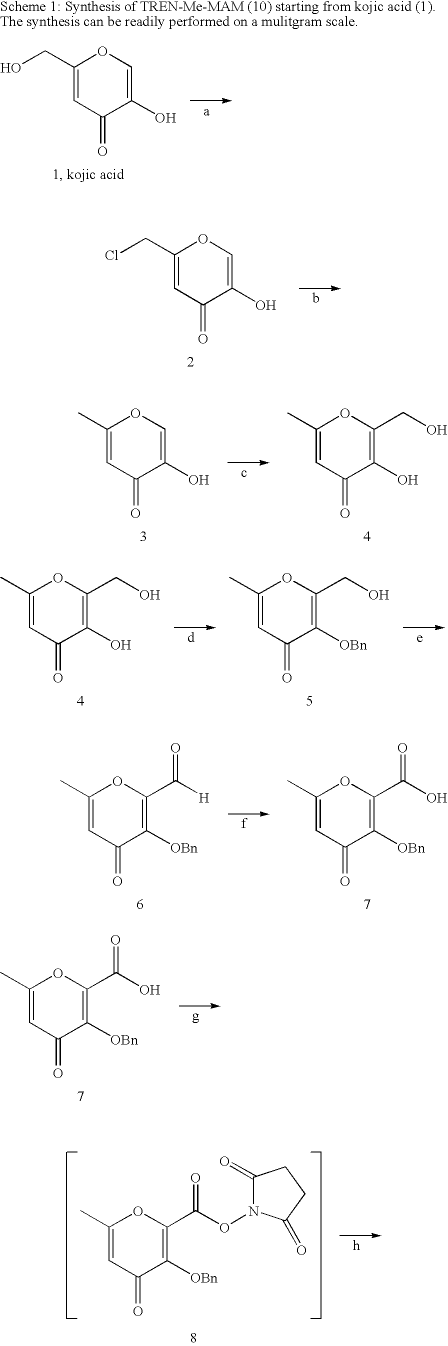

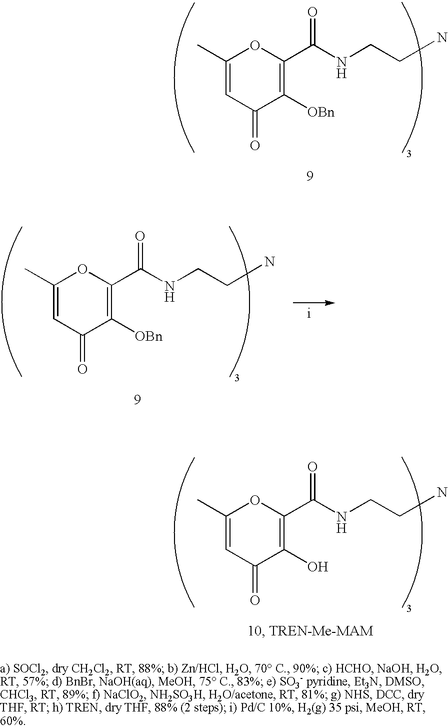

The synthesis of ligand (10), starting from commercially available kojic acid (1) is shown in scheme 1. Briefly, kojic acid (1) is dehydroxylated in two steps (a,b) to give allomaltol (3). Using formaldehyde, (3) is derivatized to give compound (4), which is then benzyl protected (step d). The resulting material (5) is then oxidized in two steps (e,f) to obtain the key intermediate (7). Carboxylic acid (7) can be readily converted to an activated ester (8) and reacted with a variety of polyamines, in this case TREN. Finally, the benzyl protecting groups of (9) are removed via catalytic hydrogenation (step i) to get the desired ligand TREN-Me-MAM (10).

A synthetic route to remove the 6-methyl substituent on the pyrone ring of TREN-Me-MAM is illustrated in Scheme 2. Commercially available maltol (11) is first protected with a benzyl group (step a). Then, an oxidation is performed using SeO₂, which directly coverts the benzyl-protected maltol (12) to aldehyde (13) (step b). The aldehyde is then converted to the carboxylic acid 14 (step c). The synthesis of TRENMAM from the key intermediate (14) is then identical to that described for TREN-Me-MAM (Scheme 1), with activation of the carboxylic acid to the NHS ester (step d), coupling to TREN (step e), and deprotection by hydrogenation (step f). The deprotection can also be achieved by reaction with acid. This straightforward synthetic procedure generates the desired ligand designated as TRENMAM (17). This ligand shows improved water solubility as as compared to the TREN-Me-MAM ligand. The synthetic procedure involved contains only six steps, and can be performed readily on a large, multigram scale. The synthesis utilizes the inexpensive, food additive maltol (11) as a starting material. This new pyrone ligand lacks the 6-methyl substituent on the pyrone ring (as found in TREN-Me-MAM). Such ligands and resulting metal complexes are more soluble in aqueous media (vide infra).

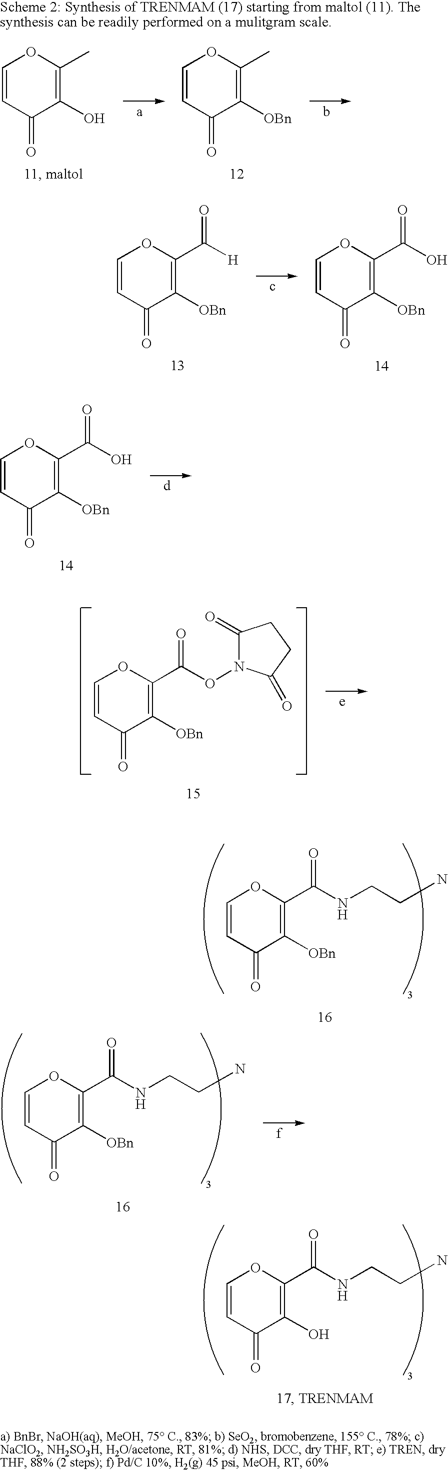

A variety of substituted pyrone compounds are readily accessible for elaboration of the TRENMAM family of ligands. Some exemplary functionalized TRENMAM derivatives are shown in Scheme 3. Each of these derivative allows for selective reactivity of each functional group to create the TRENMAM scaffold, while leaving the second site accessible for functionalization. For derivative (18), the pendant hydroxyl group can alone serve as additional solubilizing group, or be further derivatized to an ether by a variety of methods to append more complex functionalities. For precursor (19), the appended azido group readily undergoes ‘click’ chemistry, a copper-mediated coupling with an acetylene group to generate a triazole ring in high yield and with a high tolerance for other functional groups. Derivative (19) can also be reduced to an amine group for potential functionalization via imine, amide, and alkylation reactions. Finally, with derivative 20, the second carbonyl group on the ring can be selectively functionalized to couple groups via an amide or ester bond forming reaction. All three derivatives are suitable for addition of various moieties to improve solubility, relaxivity, and targeting.

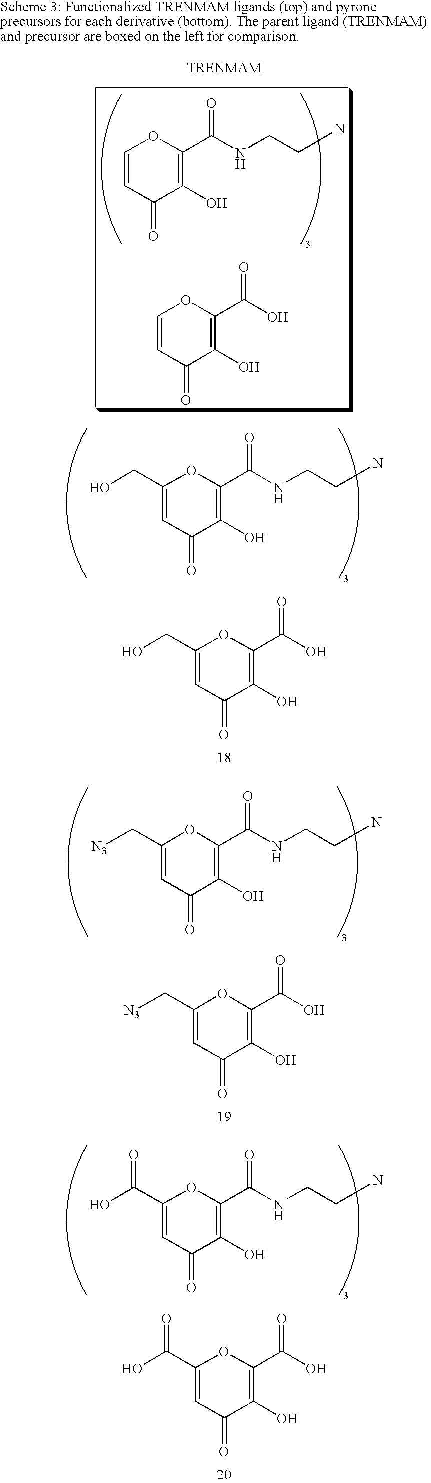

Synthetic routes for preparing compounds 18 and 20 and their TRENMAM derivatives are well outlined by literature reports. As an example, the synthetic route for 19 and some proposed derivatives are shown in Scheme 4. Starting from kojic acid (1), an azide derivative is prepared in two steps (a,b). The azide derivative (21) is then formylated and the ring hydroxyl group is protected by a blocking group, such as, benzyl group in another two steps (c,d). The compound (23) is then oxidized in two steps to get the key intermediate 19 containing a carboxylic acid. As per the synthetic schemes already described, this acid can be activated and coupled to TREN (or other polyamine backbone of interest) to obtain the protected tripodal ligand with three pendant azido groups (26). The azide group can then be used to functionalize the ligand either by using ‘click’ chemistry (route 1, step i) or reduction to the amine followed by coupling, such as by formation of an amide bond (route 2, steps j,k). Either ligand is then deprotected under standard conditions to get the final, fully functionalized TRENMAM derivatives.

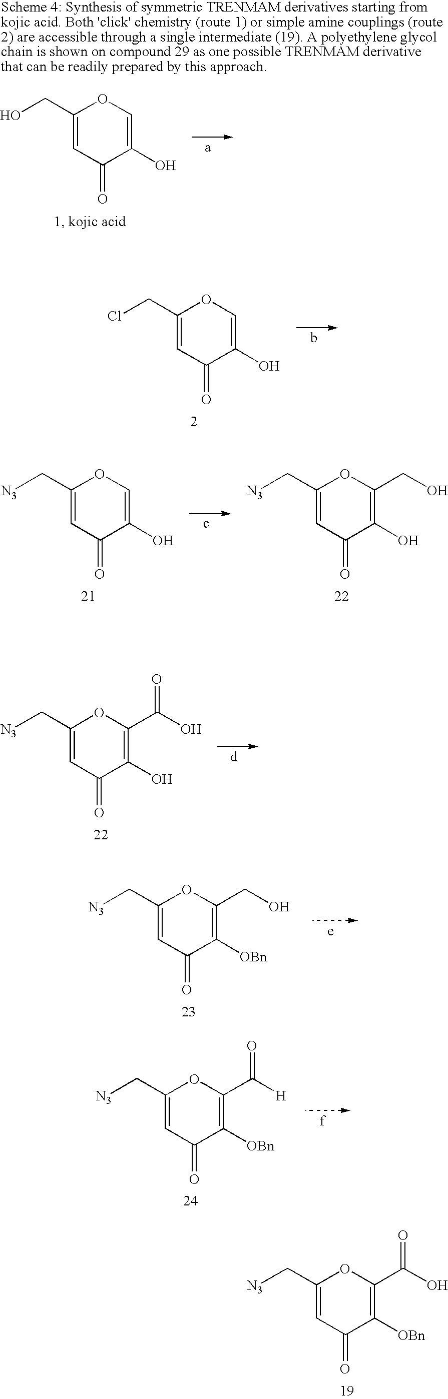

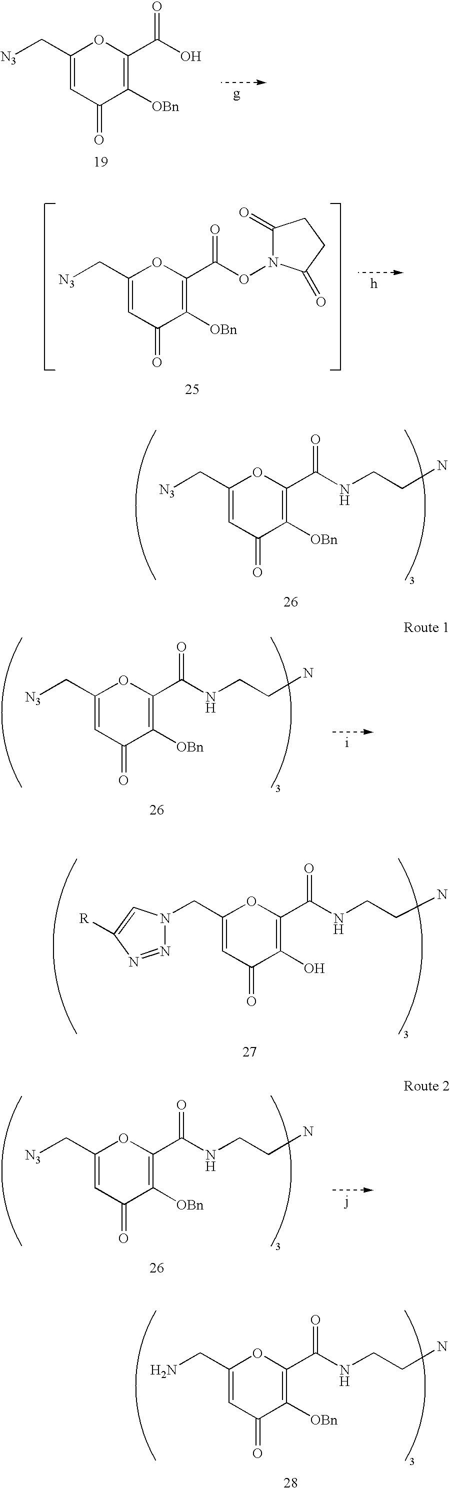

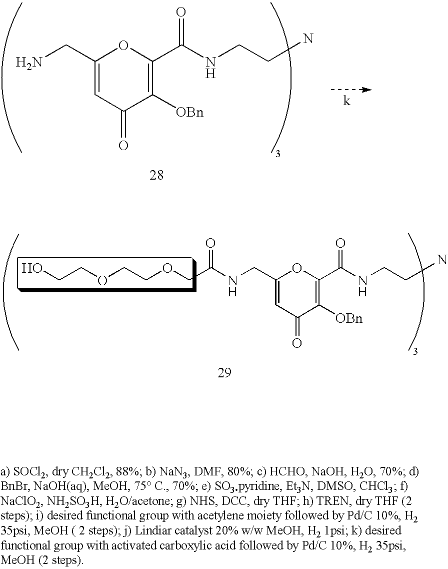

Preparation of the functionalized intermediates 18-20 can also permit synthesis of asymmetrically substituted TRENMAM derivatives as shown in Scheme 5. Asymmetrically substituted ligands can be generated by the same strategy used in previously described heteropodands.

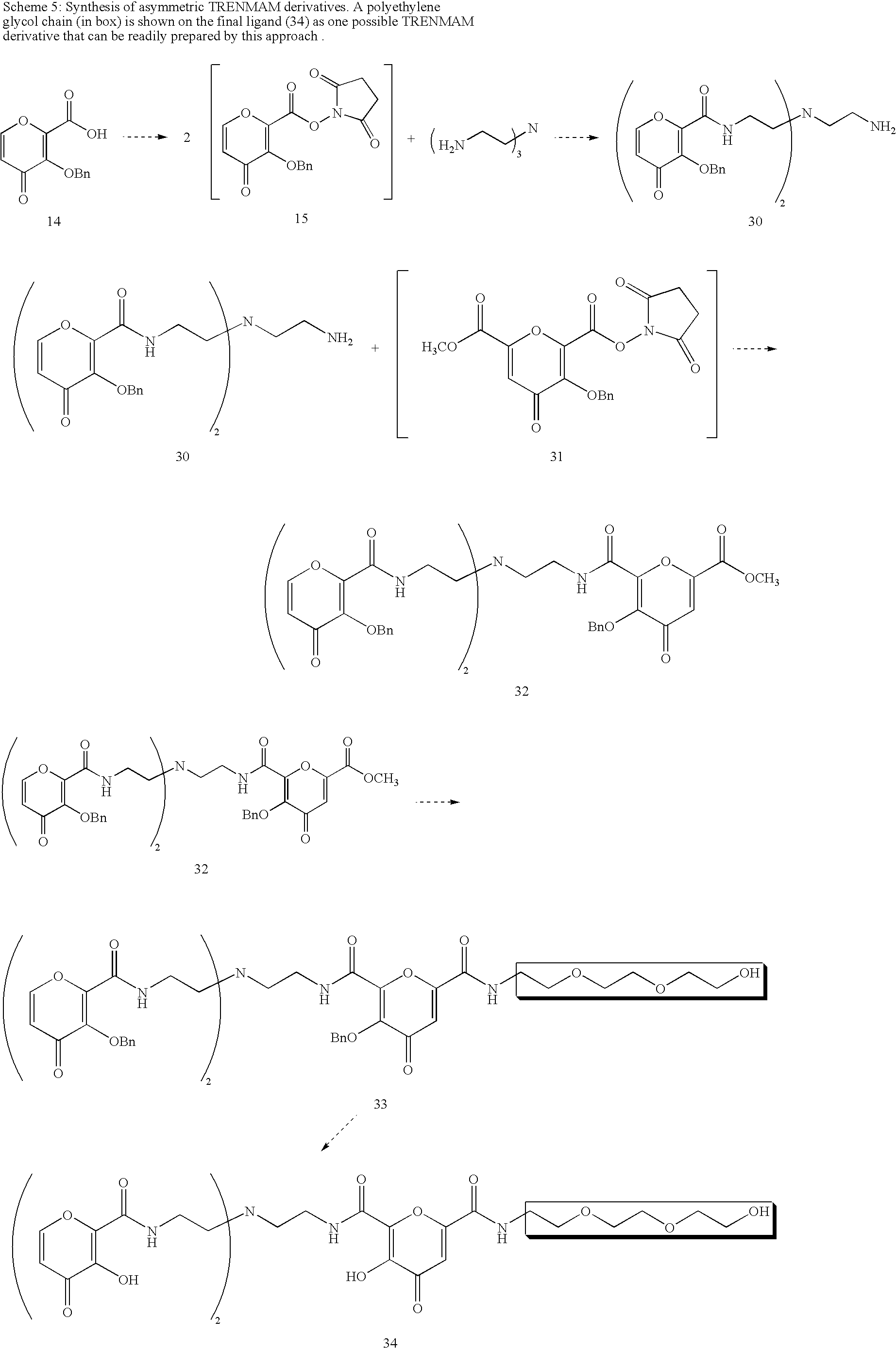

Metal complexes can be prepared by the methods described herein. Generally, the ligand can be dissolved in water or methanol, followed by addition of the appropriate metal salt (chloride, sulfate, nitrate) and a base (pyridine). In certain embodiments, the base is used in excess. The reaction mixtures are then briefly heated to reflux (˜2 h). The complexes can be isolated by direct precipitation from the reaction mixtures, or for more soluble species as found here, can be precipitated by addition of a non-polar solvent to the reaction mixture (e.g. diethylether). The complexes can also be purified by filtration and recrystallization when required.

### D. Formulation of Pharmaceutical Compositions

The pharmaceutical compositions provided herein contain therapeutically effective amounts of one or more of the compounds provided herein that are useful as MRI contrast agents and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that is useful as contrast agent. In one embodiment, the compositions are formulated for single dosage administration. To formulate a composition, the weight fraction of compound is dissolved, suspended, dispersed or otherwise mixed in a selected carrier at an effective concentration.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems well known to those of skill in the art and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, See, e.g., Remington's Pharmaceutical Sciences, 20th ed., Mack Publishing, Easton Pa. (2000). Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art. Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Controlled Release Dosage Form

The compound provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984). In certain embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

c. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

2. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

For nasal administration, the preparation may contain an esterified phosphonate compound dissolved or suspended in a liquid carrier, in particular, an aqueous carrier, for aerosol application. The carrier may contain solubilizing agents such as propylene glycol, surfactants, absorption enhancers such as lecithin or cyclodextrin, or preservatives.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm. Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Dosages

In certain embodiments, the contrast agents provided herein are administered at a dosage of 0.01-0.3 mmol/kg patient in 0.5 M solutions. The dose can be adjusted to achieve maximal efficacy in humans based on the methods well-known in the art, such as methods described in U.S. Pat. No. 6,846,915.

7. Articles of Manufacture

The compounds or pharmaceutically acceptable derivatives may be packaged as articles of manufacture containing packaging material, a compound or pharmaceutically acceptable derivative thereof provided herein, which is useful as an contrast agent, within the packaging material, and a label that indicates that the compound or composition, or pharmaceutically acceptable derivative thereof, is used as an contrast agent.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated for use as contrast agents.

### E. Evaluation of the Stability, Relaxivity and Imaging of the Compounds Evaluation of the Stability

The toxicity of uncomplexed Gd³⁺ (e.g. [Gd(H₂O)₈]³⁺) requires that any ligand designed to bind the metal ion for MRI contrast agent must be both thermodynamically and kinetically stable. It has been noted that low thermodynamic stability with Gd³⁺ will result in poor in vivo stability, and that a low selectivity for Gd³⁺ verses other metal ions is correlated with toxicity. Due to the numerous parallels between the TRENMAM and TREN-Me-3,2-HOPO ligands, the pyrone-derived ligands provided herein have thermodynamic stability (e.g. high binding constant) and selectivity (high affinity for Gd³⁺ vs. other metal ions). In order to determine the stability of [Gd(TRENMAM)], [Gd(TREN-Me-MAM)], and other derivatizes synthesized herein, a solution thermodynamics study can be performed as described herein.

The affinity of these ligands for protons and metal ions in solution is described in terms of formation constants. The protonation of a metal complex can be subsequently calculated from the appropriate cumulative constants. The stability of metal complexes can be discussed in terms of the pM value (in this case pGd) which is expressed as

pM=−log[M]free.

Although a pM value can be calculated under various conditions (e.g. different pH's or relative concentrations), in the context of a biological scenario the following parameters are commonly used for comparison: [M]=1 μM, [L]=10 μM, and pH=7.4. With these values the pM takes into account competition by protons for the ligand under physiological conditions, and has been shown to be a more accurate representation, rather than the overall formation constant, of the in vivo stability of a complex.

Thermodynamic evaluation of the new ligands can include potentiometric determination of the ligand protonation constants and spectrophotometric evaluation of the Gd³⁺, Ca²⁺, and Zn²⁺ stability constants. In order to determine the stability of these ligands with Gd³⁺, first the protonation constants for each ligand is determined. The protonation constants can be measured by potentiometric titration and the assignment of these protonations can be made using ¹H NMR titrations. ¹H NMR titration experiments use changes in the chemical shift of the non-exchangeable protons on the ligand to assign the sites of protonation; data is plotted as chemical shift vs. pD.

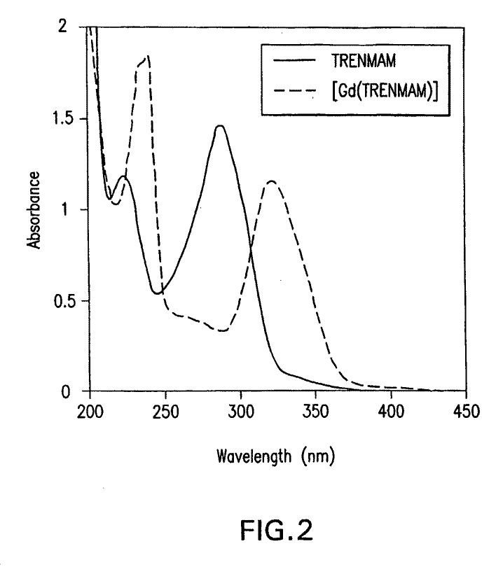

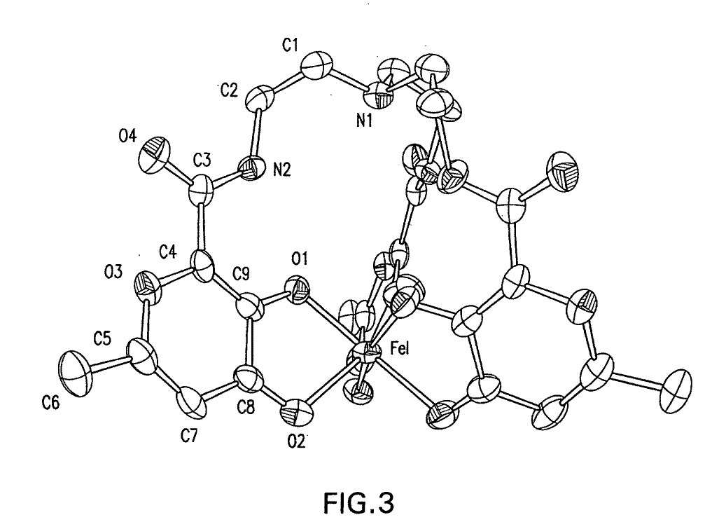

The stability of the heteropodate ligands with Gd³⁺, Ca²⁺, and Zn²⁺ can be measured by spectrophotometric titration. The strong UV absorptions (π→π*) of the pyrone rings of the ligands can be monitored in order to follow the complexation reaction (FIG. 2). The titration can be performed in a batch mode, collecting between 20-40 spectra, monitored between 200-500 nm for each titration of a ligand in the presence of an equal molar amount of Gd³⁺, Ca²⁺ or Zn²⁺. The pM can be measured by titration with a competitor ligand (DTPA or DTPA-BMA, FIG. 3), where the difference in pM for the TRENMAM complex versus the competitor ligand can be readily determined by plotting the log([MLcomp]/[ML] vs. log([Lcomp]/[L]) (where M=metal ion, Lcomp=competitor ligand, L=ligand of interest). The concentration of ML and L can be measured directly from the electronic absorption spectrum, and the pM for the TRENMAM ligand can then be determined from the known pM values of the competitor ligand. The experiments can be performed at pH 7.4, an ionic strength of 0.1 M KCl, and at 25° C. As can be seen from the spectra shown in FIG. 2, the free ligand and Gd³⁺ complex have significantly different electronic spectra, which can be monitored during the titration in order to obtain the concentration of species in solution. Analysis of the spectral data can be performed using, for example, the software package Specfit (version 3.0.36). Specfit can be used to perform factor analysis to determine the number of absorbing species in solution, to identify any species other than the free ligand and metal complex (e.g. protonated metal complexes).

A high conditional stability constant (pM value) is required to obtain a MRI-CA that is sufficiently stable in vivo to avoid toxicity associated with either free Gd³⁺ or the free ligand. Furthermore, a high degree of selectivity for Gd³⁺ over other biologically accessible metal ions, such as Ca²⁺ and Zn²⁺, has been directly correlated with in vivo toxicity. Based on these established criteria, the protonation and formation constants for the TRENMAM family of ligands can be measured in order to determine whether they are viable systems for use in MRI-CA.

Relaxivity and Imaging Evaluation of Compounds

The utility of a MRI-CA is determined by the ability of a compound to improve image quality. Certain of the parameters relevant to MRI-CA include, but are not limited to the number of inner sphere water molecules (q), the distance between the water protons and metal center (rH), the rotational correlation time (τR), and the overall relaxivity (R1). Further, in vivo imaging experiments can be performed to test the utility of the compounds as MRI-CA.

In certain embodiments, the parameters to obtain improved imaging include: a) q value>1, indicating multiple metal-bound water molecules; b) a short τM that facilitates relaxation of the bulk water by the paramagnetic center; c) a shorter distance (rH) between the metal ion and the inner-sphere water molecule; d) a longer τR that results in increased relaxivity. In certain embodiments, the compounds provided herein have a higher relaxivity than the commercially available MRI-CA (current agents are ˜5 mM−1s−1). Such compounds have utility for diagnostic purposes.

Relaxivity studies can be performed using modified 1H NMR methods. The longitudinal water proton relaxation rate can be measured, in certain embodiments, by using a Spinmaster spectrometer operating at 0.5 T; and a routine inversion-recovery technique can be employed. The 90°-pulse width can be 3.5 ms, giving reproducible T1 data. The temperature can be controlled with a Stelar VTC-91 air-flow heater equipped with a thermocouple (10.1° C.). The proton 1/T1 NMRD profiles can be measured on a Koenig-Brown field-cycling relaxometer with varying magnetic field strengths (corresponding to 0.01-70 MHz proton Larmor frequencies).

Variable-temperature ¹⁷O NMR measurements can also be performed. Measuring the transverse ¹⁷O NMR relaxation time at various temperatures allows for determination of the residence water lifetime (τM). These measurements can be recorded, for example, on JEOL EX-90 (2.1 T) and EX-400 (9.4 T) spectrometers equipped with a 5 mm probe. A D₂O external lock and solutions containing 2.6% of the ¹⁷O isotope (Yeda) can be used. The observed transverse relaxation rates can be calculated from the signal width at half height.

### F. Methods of Use of the Compounds and Compositions

The compounds and compositions provided herein are of use in a range of diagnostic imaging modalities including, but not limited to, MRI, X-ray and CT. In certain embodiments, the compounds and compositions provided herein are useful for general imaging of tumors, blood-brain-barrier breakdown, and other lesions. In addition they can be useful for examining perfusion, i.e., the blood flow into and out of tissues (heart, brain, legs, lungs, kidneys, tumors, etc.), and blood vessels (MR angiography). In certain embodiments, the compounds and compositions can be used to enhance the signal changes in the brain during cognitive events (functional MRI).

In one embodiment, provided herein are methods of enhancing tissue-specific contrast of magnetic resonance images of organs and tissues of a subject, comprising the step of administrating a diagnostically effective amount of a compound provided herein.

In certain embodiment, the contrast agents provided herein are used to enhance diagnostic X-ray imaging as well as ultrasound and light imaging. In these cases, the doses of the agent will be approximately equal to that in MRI (0.001-10 mmol/kg). For nuclear imaging, however, the doses will be at tracer levels. For all of these techniques, the use and administration of contrast agents and the settings on the imaging machines is known in the art or uses commonly accepted principles.

In an exemplary embodiment, provided herein is a method for performing a contrast enhanced imaging study on a subject. The method includes administering a metal complex provided herein to the subject and acquiring an image of the subject.

In certain embodiments, provided herein are method for performing a contrast enhanced imaging study on a subject comprising administering a compound provided herein to the subject and acquiring an MRI of the subject.

In certain embodiments, the compounds provided herein can be used in combination with a second contrast agent. Several contrast agents are known to one of skill in the art, for example, see, U.S. Pat. Nos. 6,846,915 and 6,676,929.

The following examples are included for illustrative purposes only and are not intended to limit the scope of the subject matter claimed herein.

### EXAMPLES

Unless otherwise noted, starting materials were obtained from commercial suppliers and used without further purification. Elemental analysis was performed at NuMega Resonance Labs, San Diego, Calif. 1H/13C NMR spectra were recorded on a Varian FT-NMR spectrometer running at 300 or 400 MHz at the Department of Chemistry and Biochemistry, University of California, San Diego. Mass spectra were acquired at the Small Molecule Mass Spectrometry Facility located in the Department of Chemistry and Biochemistry, University of California, San Diego.

### Example 1

### Preparation of TRENMAM

The title compound was prepared as illustrated in Scheme 2

### Preparation of Protected-TRENMAM

2-Formyl-3-benzyloxy-pyran-4(1H)-one was synthesized from maltol (3-hydroxy-2-methyl-4-pyrone) according to a literature procedure (Pace P. et al.; Bioorg. Med. Chem. Lett. 2004, 14, 3257). 2-Carboxy-3-benzyloxy-pyran-4(1H)-one) was synthesized using an analogous procedure for the synthesis of 2-carboxy-3-benzyloxy-6-methyl-pyran-4(1H)-one as described by a literature procedure (Liu, Z. et al., Bioorg. Med. Chem. 2001, 9, 563). To a suspension of 2-Carboxy-3-benzyloxy-pyran-4(1H)-one) (3.0 g, 12.2 mmol) in dry THF (90 mL) was added NHS (1.4 g, 12.2 mmol) and the mixture was stirred at room temperature under N₂(g) for 30 min. DCC (2.52 g, 12.2 mmol) was then added and the mixture was stirred at room temperature under N₂(g) for 3 h. The DCU precipitate was removed by filtration, and to the resulting filtrate was added a solution of tris(2-aminoethyl)amine (TREN, 607.5 μL, 4.1 mmol) in THF (10 mL) dropwise over 15 min. The reaction mixture was stirred overnight at room temperature under N₂(g). A white precipitate was filtered off and the filtrate was evaporated to dryness to obtain an amber oil. The oil was dissolved in CHCl₃. The CHCl₃ solution was washed with 2×150 mL of saturated NaHCO₃. The organic layer was dried over MgSO₄ and filtered. The filtrate was evaporated to obtain an amber oil. The oil was purified by silica column chromatography (CHCl₃ with 0-9% MeOH) to yield a foamy off-white solid (2.7 g, 80%).

¹H NMR (CDCl₃, 300 MHz, 25° C.): δ 2.30 (t, J=6.4 Hz, 6H, CH₂), 3.12 (q, J=6.0 Hz, 6H, CH₂), 5.33 (s, 6H, benzyl-CH₂), 6.46 (d, J=6.0 Hz, 3H, pyrone-H), 7.34 (s, 15H, benzyl-H), 7.69 (t, J=5.4 Hz, 3H, amide-H), 7.82 (d, J=5.4 Hz, 3H, pyrone-H). ¹³C NMR (CDCl₃, 100 MHz, 25° C.): δ 25.5, 36.6, 52.4, 75.3, 117.4, 128.7, 129.0, 135.2, 146.8, 154.6, 159.4, 172.3, 175.6. ESI-MS (+): m/z 853.18 [M+Na]+.

### Preparation of TRENMAM

The protected TRENMAM obtained above, (500 mg, 0.6 mmol) was added 13.5 mL of a 1:1 solution of concentrated HCl and glacial acetic acid. The suspension was stirred under N₂(g) for 24 h at room temperature. The reaction was co-evaporated with methanol (3×20 mL) and dried under vacuum to yield a white solid (280 mg, 83%).

¹H NMR (d-DMSO, 400 MHz, 25° C.): δ 3.40 (br t, 6H, CH₂), 3.69 (br q, 6H, CH₂), 6.46 (d, J=5.2 Hz, 3H, pyrone-H), 8.15 (d, J=5.6 Hz, 3H, pyrone-H), 8.92 (t br, 3H, amide-H), 10.25 (s br, 1H, OH), 11.20 (s br, 2H, OH). ¹³C NMR (d-DMSO, 100 MHz, 25° C.): δ 51.1, 114.5, 136.7, 148.1, 154.8, 162.4, 173.2. ESI-MS (+): m/z 561.10 [M+H]+. Anal. Calcd for C₂₄H₂₄N₄O₁₂.2.5H₂O: C, 47.61; H, 4.83; N, 9.25. Found: C, 47.85; H, 4.83; N, 8.81.

### Example 2

### Preparation of TREN-Me-MAM

The title compound was prepared from as illustrated in Scheme 1.

### Preparation of Protected-TREN-Me-MAM

Protected-TREN-Me-MAM was synthesized in a similar manner but starting from 2.0 g (7.6 mmol) of 2-carboxy-3-benzyloxy-6-methyl-pyran-4(1H)-one (Puerta, D. T. et al. J. Am. Chem. Soc. 2005, 127, 14148). The product was isolated as a white foamy solid (2.0 g, 90%).

¹H NMR (CDCl₃, 300 MHz, 25° C.): δ 2.30 (t, J=6.6 Hz, 6H, CH₂), 2.36 (s, 9H, methyl-H), 3.10 (q, J=6.0 Hz, 6H, CH₂), 5.33 (s, 6H, benzyl-CH₂), 6.26 (s, 3H, pyrone-H), 7.31 (s, 15H, benzyl-H), 7.70 (t, J=5.4 Hz, 3H, amide-H). ¹³C NMR (CDCl₃, 100 MHz, 25° C.): δ 25.5, 36.8, 52.3, 75.3, 79.1, 117.4, 128.7, 129.1, 135.2, 146.8, 154.6, 159.0, 172.3, 175.7. ESI-MS (+): m/z 895.20 [M+Na]+.

Preparation of TREN-Me-MAM

Protected-TREN-Me-MAM (1.8 g, 2.1 mmol) was dissolved in methanol (100 mL). To this solution was added 110 mg 10% Pd/C and the mixture was placed under H₂(g) at 35 psi for 16 h. The Pd catalyst was removed by filtration and the filtrate was evaporated to a white solid (1.1 g, 89%).

¹H NMR (d-DMSO, 400 MHz, 25° C.): δ 2.25 (s, 9H, methyl-H), 2.71 (br t, 6H, CH₂), 3.35 (br q, 6H, CH₂), 6.24 (d, J=5.2 Hz, 3H, pyrone-H), 8.61 (t br, 3H, amide-H). ¹³C NMR (d-DMSO, 100 MHz, 25° C.): δ 19.4, 37.3, 52.5, 112.4, 136.0, 147.1, 162.1, 164.4, 173.3. IR (KBr pellet): ν 1233, 1352, 1443, 1553, 1653, 3411 cm⁻¹. ESI-MS (+): m/z 625.14 [M+Na]⁺. Anal. Calcd for C₂₇H₃₀N₄O₁₂.2H₂O: C, 50.78; H, 5.37; N, 8.77. Found: C, 50.48; H, 5.53; N, 8.72.

The protonation constants for TRENMAM and TREN-ME-MAM are provided below:

Protonation Constants for Pyrone Ligands as Measured by Potentiometric Titrations:

| Constant | TRENMAM | TREN-Me-MAM |
|---|---|---|
| log K₁ | 7.33(1) | 7.91(1) |
| log K₂ | 5.76(1) | 6.30(2) |
| log K₃ | 4.97(2) | 5.48(2) |
| log K₄ | 3.84(2) | 4.46(2) |

### Example 3

### Preparation of [Gd(TRENMAM)]

TRENMAM (150 mg, 0.27 mmol) was dissolved in hot methanol (100 mL) and water (50 mL). Gd(NO₃)₃.5H20 (110 mg, 0.25 mmol) was added to the hot solution, followed by an excess of pyridine. The reaction mixture was heated to reflux for 2 h. The reaction mixture was evaporated to dryness giving an off-white solid. The solid was washed with a minimal amount of methanol and dried to yield an off-white solid (180 mg, 94%). ESI-MS (+): m/z 716.03 [M+H]+.

### Example 4

### Preparation of [Gd(TREN-Me-MAM)]

TREN-Me-MAM (100 mg, 0.17 mmol) was dissolved in ethanol (15 mL), and Gd(NO₃)₃.5H₂O (72 mg, 0.17 mmol) was added to the solution, which instantly precipitated a white solid. To this suspension was added an excess of pyridine. The reaction mixture was heated to reflux for 2 h, followed by hot filtration of the solid. The white solid was washed with a minimal amount of cold ethanol and dried (90 mg, 72%). IR (KBr pellet): v 1250, 1384, 1454, 1552, 1602, 3419 cm⁻¹. ESI-MS (+): m/z 780.07 [M+Na]+.

[Gd(TREN-Me-MAM)] is found to have an aqueous solubility in pH 7 water of >100 mM, which is greater than found for [Gd(TRENMAM)].

### Example 5

### Preparation of [Fe(TREN-Me-MAM)]

TREN-Me-MAM (100 mg, 0.17 mmol) was dissolved in methanol (20 mL), and FeCl₃.6H₂O (45 mg, 0.17 mmol) was added, followed by the addition of an excess of pyridine. The resulting red suspension was heated at reflux for 2 h. The reaction mixture was evaporated to dryness and sonicated in isopropanol, filtered, and dried. The complex was further purified by silica column chromatography (CHCl₃ with 3% MeOH) to yield a red solid (95 mg, 87%). ESI-MS (+): m/z 678.11 [M+Na]+.

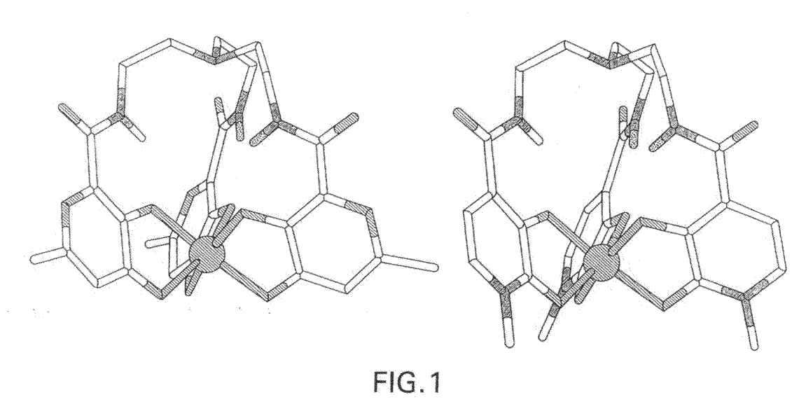

FIG. 1 compares the structure of [Fe(TREN-Me-MAM)] to that of [Fe(TREN-Me-3,2-HOPO)]. Both complexes display three intramolecular hydrogen bonds between the amide protons of each ‘arm’ and the deprotonated phenolate oxygen atom coordinated to the iron center (FIG. 1). The complex [Fe(TREN-Me-3,2-HOPO)] is insoluble in water, while [Fe(TREN-Me-MAM)] has good aqueous solubility (˜20 mM in neutral water).

### Example 6

### X-Ray Crystallographic Analysis

Red cubes of [Fe(TREN-Me-MAM)] suitable for X-ray diffraction structural determination were grown by slow evaporation from chloroform. Data were collected on a Bruker AXS area detector diffractotheter. Crystals were mounted on quartz capillaries by using Paratone oil and were cooled in a nitrogen stream (Kryo-flex controlled) on the diffractometer (−173° C.). Peak integrations were performed with the Siemens SAINT software package. Absorption corrections were applied using the program SADABS. Space group determinations were performed by the program XPREP. The structures were solved by direct methods and refined with the SHELXTL software package (Sheldrick, G. M. SHELXTL vers. 5.1 Software Reference Manual; Bruker AXS: Madison, Wis., 1997). All hydrogen atoms were fixed at calculated positions with isotropic thermal parameters; all non-hydrogen atoms were refined anisotropically. (CCDC deposition number 289563).

TABLE 1
Summary of X-ray structural parameters for [Fe(TREN-Me-MAM)].

| | |
|---|---|
| Emp. Formula | C27H27N4O12Fe |
| Crystal System | Cubic |
| Space Group | P21/c |
| Unit Cell dimensions | a = b = c = 17.6481(5) Å |
| Volume, Z | 5496.6(3) Å3, 8 |
| Crystal size | 0.20 × 0.20 × 0.20 mm3 |
| Temperature (K) | 100(2) |
| Reflections collected | 48183 |
| Independent reflections | 4236 [R(int) = 0.0669] |
| Data/rest./para. | 4236/0/267 |
| Goodness-of-fit on F2 | 1.057 |
| Final R indices I > 2σ(I)ₐ | R1 = 0.0556 |
| wR2 = 0.1313 | |
| R indices (all | R1 = 0.0715 |
| data)ₐ | |
| wR2 = 0.1397 | |
| Largest peak/hole difference | 0.837/−0.437 e Å−3 |

### Example 7

### Solution Thermodynamics

The experimental protocols and equipment used have been previously described (Johnson, A. R., et al. Inorg. Chem. 2000, 39, 2652). To determine the protonation constants of the free ligands, approximately 15 mg of ligand was dissolved in 50 mL of a 1.0 M aqueous solution of NaCl in a titration vessel (ligand concentration ˜0.5 mM). Protonation constants of TRENMAM and TREN-Me-MAM were examined by potentiometric (pH vs total proton concentration) titrations by using Hyperquad (Gans, P. et al. Talanta 1996, 43, 1739) for data analysis. Each protonation constant determination is the result from at least three experiments (where each experiment consists of two titrations, the first one titrated with acid, followed by a reverse titration with base). The equilibration time between additions of titrant was 90 seconds.

To determine the formation constants of [Gd(TRENMAM)], the same solutions and equipment were used as in the determination of the protonation constants of the free ligands. Approximately 12 mg of TRENMAM was dissolved in 50 mL of a 1.0 M aqueous solution of NaCl, followed by the addition of an equimolar amount of aqueous GdCl₃ solution (TRENMAM and Gd concentration ˜0.4 mM). The solution was acidified to a pH of 2.5 with 0.1 M HCl and the resulting solution was then titrated with 0.1 M NaOH in 0.05 mL increments to a final pH of 11. The [Gd(TRENMAM)(H₂O)₂] complex did not fully disassociate under these conditions, therefore a titration at lower pH (1.6-2.5) was also performed. For these measurements, a correction of the liquid junction potential for low pH was performed in the course of pH electrode calibration as in former work (Johnson, A. R. et al. Inorg. Chem. 2000, 39, 2652).

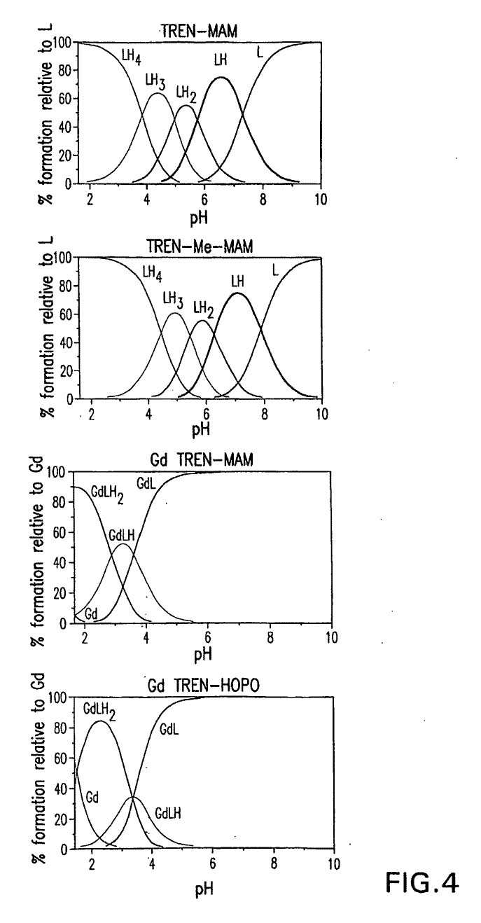

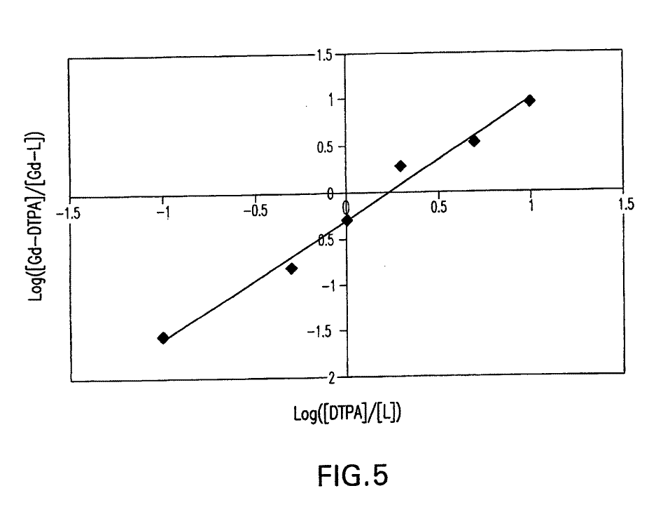

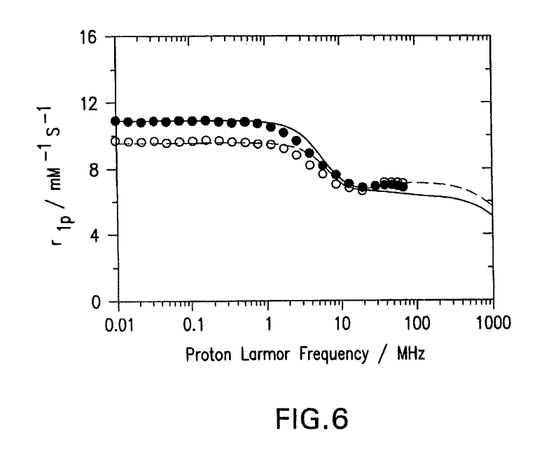

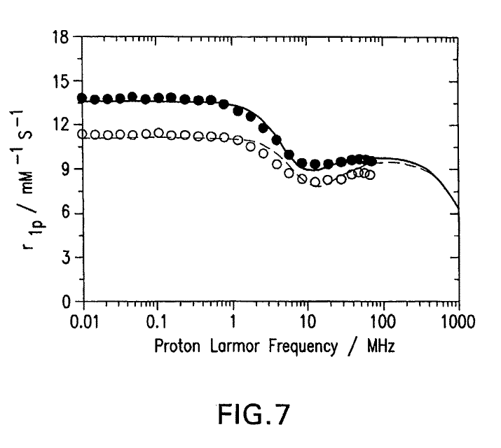

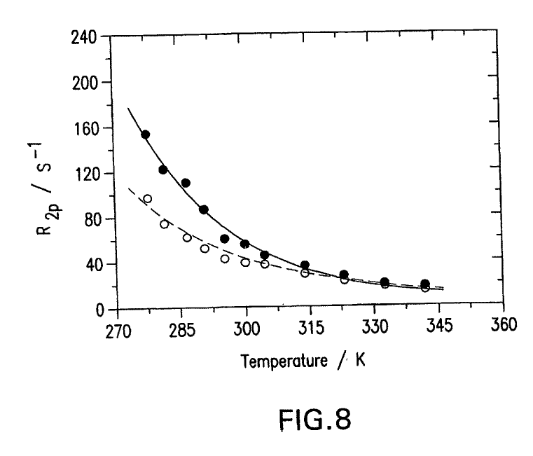

The titrations at a lower pH still failed to fully disassociate the complex due to the acidic nature of the ligand (FIG. 4). Therefore, a β110 could not be determined and was calculated from a competition titration with DTPA (vide infra). The other formation constants, β111 and β112, were determined from experiments (where each experiment consists of two titrations, the first one titrated with acid, followed by a reverse titration with base). The refinement of two experiments between pH=2.5 10.5 and six titrations at low pH from pH=1.6 2.5, using a junction potential calibration (as described in Johnson, A. R. et al. Inorg. Chem. 2000, 39, 2652) with fixed ligand protonation constants and a fixed β110 gave β111=22.15(9) and β112=25.2(2) for [Gd(TRENMAM)]. The equilibration time between additions of titrant was 300 seconds.

### Example 8

### Competition Titration with DTPA

The general procedure used to determine the pGd of [Gd(TRENMAM)] and [Gd(TREN-Me-MAM)] by competition batch titration was adapted from a previous report (Doble, D. M. J et al. Inorg. Chem. 2003, 42, 4930). Varying volumes of a standardized DTPA stock solution were added to solutions containing constant ligand, metal, and electrolyte concentrations. The pH of all solutions was adjusted to 7.4 with HCl and/or KOH and the solutions were diluted to identical volumes. The concentrations of TRENMAM or TREN-Me-MAM relative to DTPA used in the final data analysis ranged from 1:0.1 to 1:10 (TRENMAM:DTPA or TREN-Me-MAM:DTPA). After stirring for 24 h to ensure thermodynamic equilibrium, concentrations of free and complexed TRENMAM or TREN-Me-MAM were determined from the absorption spectra, using the wavelength range of 335-370 nm and the spectra of free and fully complexed TRENMAM or TREN-Me-MAM at identical pH and concentrations as references for the analysis. Three titrations were performed, resulting in a pGd of 19.27±0.08 and 19.03±0.04 for TRENMAM (FIG. 5) and TREN-Me-MAM, respectively.

### Example 9

### Relaxivity Data for Gd³⁺ Complexes of TREN-Me-MAM and TRENMAM

Relaxivity measurements for Gd³⁺ complexes of TREN-Me-MAM and TRENMAM conducted at 20 MHz and 298 K, and the data is provided below:
Complex Relaxivity
Gd(TRENMAM)(H₂O)₂ 9.3 mM⁻¹s⁻¹
Gd(TREN-Me-MAM(H₂O)₂ 8.2 mM⁻¹s⁻¹

These values are constant in the pH range of 4-9. A detailed NMRD (at 298 and 310 K, FIG. 6) and variable temperature ¹⁷O NMR (at 2.12 T, FIG. 8) study. FIG. 7 shows the 1/T₁ NMRD profiles of the two complexes recorded at 298 K over the frequency range of 0.01-70 MHz. A simultaneous fitting of both the NMRD and ¹⁷O NMR data provided the parameters listed in Table 2.

TABLE 2
Parameters Obtained from the Simultaneous Fitting of
¹H NMRD and ¹⁷O NMR Data

| | [Gd(TRENMAM)] | [Gd(TREN-Me-MAM)] |
|---|---|---|
| Δ²/10⁹ s⁻² | 5.6 ± 0.3 | 10.8 ± 0.2 |
| ²⁹⁸τᵥ/ps | 19.0 ± 0.8 | 15.2 ± 1.2 |
| ²⁹⁸τ_{R}/ps | 145 ± 6 | 120 ± 9 |
| ²⁹⁸τ_{M}/ns | 1.1 ± 0.3 | 1.0 ± 0.4 |
| ΔH_{M}/kJ mol⁻¹ | 27.6 ± 0.9 | 22.4 ± 1.8 |
| Eᵥ/kJ^{a} | 1 | 1 |
| r_{GdH}/Å | 3.09 ± 0.2 | 3.04 ± 0.3 |
| r_{GdO}/Å^{a} | 2.48 | 2.48 |
| A/h/10⁶ rad · s⁻¹ | −3.6 ± 0.2 | −3.8 ± 0.1 |
| q^{a} | 2 | 2 |
| a/Å^{a} | 4.0 | 4.0 |
| D/10⁻⁵ cm² s⁻¹ | 2.27 ± 0.3 | 2.30 ± 0.2 |
| E_{D}/kJ mol^{-1a} | 22 | 22 |

^{a}Values were fixed in the fitting procedure.

The complexes [Gd(TRENMAM)(H₂O)₂] and [Gd(TREN-Me-MAM)(H₂O)₂] show a fast rate of water exchange (298 kₑₓ˜8×10⁸ s−1). In certain embodiments, the very rapid water exchange kinetics is useful for MRI-CA applications at high fields (80-100 MHz), where the optimal τ_{M} values for achieving high relaxivities are close to 1 ns⁸. The relaxation rate of the complexes were measured versus concentration in the range of

0.5-100 mM (at 0.1 MHz and 298 K). In certain embodiments, 0.1 M represents the lower limit of the solubility of the complexes.

Since modifications will be apparent to those of skill in the art, it is intended that the claimed subject matter be limited only by the scope of the appended claims.

What is claimed is:

1. A compound of formula: 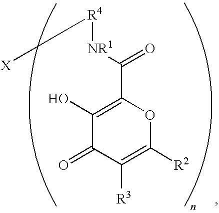
wherein X is a scaffold; n is 1-6;
R¹ is hydrogen, alkyl, alkenyl, alkynyl, aryl, heteroaryl, cycloalkyl, heterocyclyl or C(A)R⁵;
R² and R³ are each independently selected from hydrogen, alkyl, alkenyl, alkynyl, aryl, heteroaryl, cycloalkyl, heterocyclyl, C(A)R⁵, OR⁶ and NR⁷R⁸;
R⁴ is alkylene, alkenylene, alkynylene, cycloalkylene, arylene, heteroarylene or heterocyclylene group;
A is O, S or NR⁷;
R⁵ is hydrogen, alkyl, alkenyl, alkynyl, aryl, heteroaryl, heteroarylium, cycloalkyl, heterocyclyl, halo, pseudohalo, OR⁶ or NR⁷R⁸;
R⁶ is hydrogen, alkyl, alkenyl, alkynyl, aryl, heteroaryl, heteroarylium, cycloalkyl, or heterocyclyl;
R⁷ and R⁸ are selected as follows:
i) R⁷ and R⁸ are each independently selected from hydrogen, alkyl, alkenyl, alkynyl, aryl, heteroaryl, heteroarylium, cycloalkyl, and heterocyclyl; or
ii) R⁷ and R⁸ together with the nitrogen atom on which they are substituted form a heterocyclyl or heteroaryl ring;
wherein R¹-R⁸ are each independently unsubstituted or substituted with one or more substituents, each independently selected from Q¹;
where Q¹ is hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano, or isothiocyano, and
each Q¹ is independently unsubstituted or substituted with one or more substituents, each independently selected from Q²;
each Q² is independently hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano or isothiocyano.

2. The compound of claim 1, wherein R³ and R² are selected from hydrogen, alkyl and carboxy.

3. The compound of claim 1 or 2, wherein R³ is hydrogen.

4. The compound of claim 1 or 2, wherein R² is hydrogen, alkyl or carboxy.

5. The compound of any of claims 1-2 and 4, wherein R² is hydrogen, hydroxyalkyl, azidoalkyl or carboxy.

6. The compound of any of claims 1-2 and 4-5, wherein R² is hydrogen, methyl, hydroxymethyl, azidomethyl or carboxy.

7. The compound of any of claims 1-6, the compound has formula III:

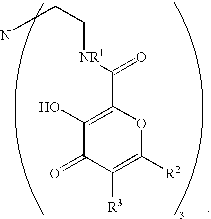

8. The compound of any of claims 1-6, wherein the compound is:

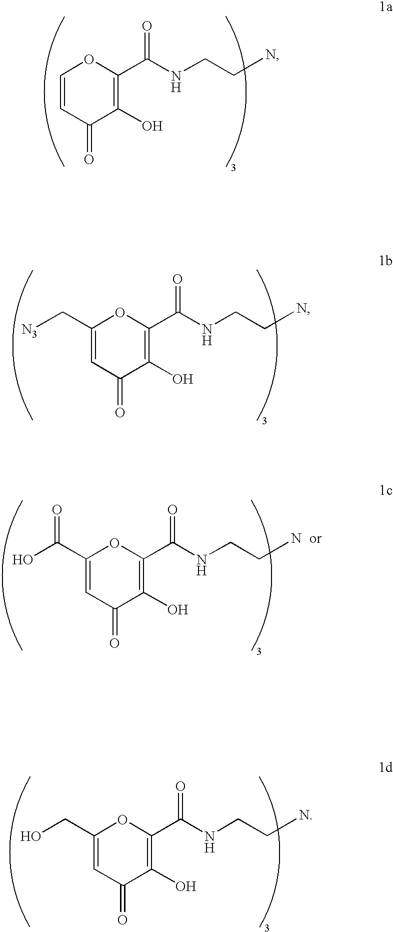

9. A compound of formula: 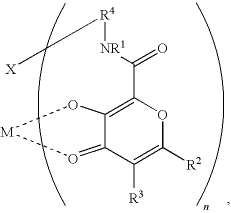
where M is selected from Gd, Ga, Dy, Fe, Mn, Pu, and U;
X is a scaffold; n is 1-6;
R¹ is hydrogen, alkyl, alkenyl, alkynyl, aryl, heteroaryl, cycloalkyl, heterocyclyl or C(A)R⁵;
R² and R³ are each independently selected from hydrogen, alkyl, alkenyl, alkynyl, aryl, heteroaryl, cycloalkyl, heterocyclyl, C(A)R⁵, OR⁶ and NR⁷R⁸;
R⁴ is alkylene, alkenylene, alkynylene, cycloalkylene, arylene, heteroarylene or heterocyclylene group;
A is O, S or NR⁷;
R⁵ is hydrogen, alkyl, alkenyl, alkynyl, aryl, heteroaryl, heteroarylium, cycloalkyl, heterocyclyl, halo, pseudohalo, OR⁶ or NR⁷R⁸;
R⁶ is hydrogen, alkyl, alkenyl, alkynyl, aryl, heteroaryl, heteroarylium, cycloalkyl, or heterocyclyl;
R⁷ and R⁸ are selected as follows:
i) R⁷ and R⁸ are each independently selected from hydrogen, alkyl, alkenyl, alkynyl, aryl, heteroaryl, heteroarylium, cycloalkyl, and heterocyclyl; or
ii) R⁷ and R⁸ together with the nitrogen atom on which they are substituted form a heterocyclyl or heteroaryl ring;
wherein R¹-R⁸ are each independently unsubstituted or substituted with one or more substituents, each independently selected from Q¹;
where Q¹ is hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano, or isothiocyano, and
each Q¹ is independently unsubstituted or substituted with one or more substituents, each independently selected from Q²;
each Q² is independently hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano or isothiocyano.

10. The compound of claim 9, wherein the compound has formula:

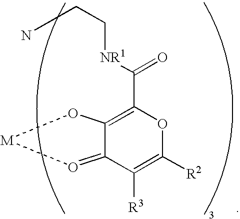

11. The compound of claim 9 or 10, wherein M is selected from Gd, Ga, and Fe.

12. The compound of any of claims 9-11, wherein the compound has formula:

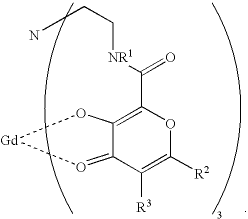

13. The compound of any of claims 9-12, wherein the compound has formula:

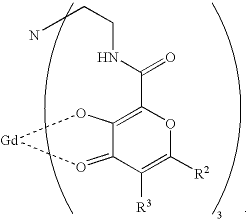

14. The compound of any of claims 9-13, wherein the compound has formula:

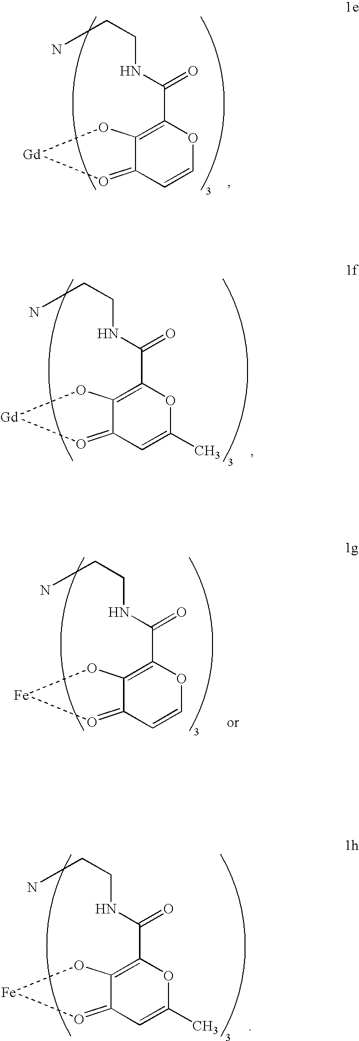

15. A compound of formula: 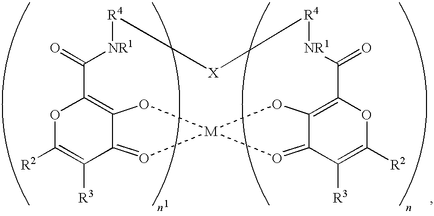
where M is selected from Gd, Ga, Dy, Fe, Mn, Pu, and U;
X is a scaffold; n and n¹ are each independently 1-6;
R¹ is hydrogen, alkyl, alkenyl, alkynyl, aryl, heteroaryl, cycloalkyl, heterocyclyl or C(A)R⁵;
R² and R³ are each independently selected from hydrogen, alkyl, alkenyl, alkynyl, aryl, heteroaryl, cycloalkyl, heterocyclyl, C(A)R⁵, OR⁶ and NR⁷R⁸;
R⁴ is alkylene, alkenylene, alkynylene, cycloalkylene, arylene, heteroarylene or heterocyclylene group;
A is O, S or NR⁷;
R⁵ is hydrogen, alkyl, alkenyl, alkynyl, aryl, heteroaryl, heteroarylium, cycloalkyl, heterocyclyl, halo, pseudohalo, OR⁶ or NR⁷R⁸;
R⁶ is hydrogen, alkyl, alkenyl, alkynyl, aryl, heteroaryl, heteroarylium, cycloalkyl, or heterocyclyl;
R⁷ and R⁸ are selected as follows:
i) R⁷ and R⁸ are each independently selected from hydrogen, alkyl, alkenyl, alkynyl, aryl, heteroaryl, heteroarylium, cycloalkyl, and heterocyclyl; or
ii) R⁷ and R⁸ together with the nitrogen atom on which they are substituted form a heterocyclyl or heteroaryl ring;
wherein R¹-R⁸ are each independently unsubstituted or substituted with one or more substituents, each independently selected from Q¹;
where Q¹ is hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano, or isothiocyano, and
each Q¹ is independently unsubstituted or substituted with one or more substituents, each independently selected from Q²;
each Q² is independently hydrogen, halo, pseudohalo, hydroxy, oxo, thia, nitrite, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano or isothiocyano.

16. A pharmaceutical composition comprising the compound of any of claims 1-15 and a pharmaceutically acceptable carrier.

17. A method for performing a contrast enhanced imaging study on a subject comprising administering a compound of any of claims 9-15 to the subject and acquiring the contrast enhanced image of the subject.

18. The method of claim 17, wherein the image is a magnetic resonance image.

19. A method of enhancing tissue-specific contrast of magnetic resonance images of organs and tissues of a subject, comprising the step of administrating the compound of any of claims 9-15.

20. The method of any of claims 17-19, further comprising administering a second contrast agent.

21. A compound of any of claims 9-15 for use in diagnostic imaging.

22. A use of a compound of any of claims 9-15 for in manufacture of a medicament for diagnostic imaging.

23. An article of manufacture, comprising packaging material and a compound of any of claims 9-15 contained within the packaging material, wherein the compound is for performing a contrast enhanced imaging study and the packaging material includes a label that indicates that the compound is used for performing a contrast enhanced imaging study.
